(19) United States
(12) Patent Application Publication
(10) Pub. No.: US 2014/0330519 A1
(43) Pub. Date: Nov. 6, 2014

(54) Method to identify multivariate anomalies by computing similarity and dissimilarity between entities and considering their spatial interdependency

(71) Applicant: Heiko Mueller, Whitehorse (CA)

(72) Inventor: Heiko Mueller, Whitehorse (CA)

(21) Appl. No.: 14/267,890

(22) Filed: May 1, 2014

Related U.S. Application Data
(60) Provisional application No. 61/818,073, filed on May 1, 2013.

(51) Int. Cl. G01N 33/24

(52) U.S. Cl. CPC G01N 33/24
USPC 702/2

(57) ABSTRACT

A method is presented for identifying anomalies based on the dissimilarity and similarity between multivariate samples. A step like procedure applies Dissimilarity- and Similarity computation in a sequenced fashion that considers variable variance, variable correlation and variable distribution pattern of the samples. The spatial interdependency of samples is assessed to deduce the nature of the anomaly. Similarity computation of samples is used to identify weak anomalies that are difficult to detect by conventional exploration methods.

### CROSS-REFERENCE TO RELATED APPLICATIONS

Not applicable

### STATEMENT REGARDING FEDERALLY SPONSORED RESEARCH OR DEVELOPMENT (IF APPLICABLE)

Not applicable

### BACKGROUND OF THE INVENTION

1. Field of the Invention

The present invention is in the technical field of chemistry. More particularly, the present invention is in the technical field of geochemical exploration. One embodiment of the invention among others relates to geochemical exploration for the analysis of rock, soil, sediment and organic matter to determine ore sources.

2. Description of the Prior Art

Sources of mineralization are recognized by anomalous element concentrations and/or abnormal element distribution pattern in rock-, soil-, sediment-, organic matter samples collected downslope of the source. Mineralization is a multi-element affair; therefore a multivariate interpretation in a spatial context is required. In general: “The basic theme underlying the use of multivariate methods in survey investigations is simplification, e.g., reducing a large and possibly complex body of data to a few meaningful summary measures or identifying key features and any interesting patterns in the data. The aim is often exploration: such methods can help in generating hypotheses of interest to the researcher rather than in testing them.” [Ref. 1, p. 3, paragraph 7]

An overview about methods in geochemical exploration is presented in: The Interpretation of Regional Geochemical Survey Data by Grunsky, E. C. in “Proceedings of Exploration 07: Fifth Decennial International Conference on Mineral Exploration” edited by B. Milkereit, 2007, p. 139-182. State of the art conventional exploration operates on one of the three separate approaches. These are:
A. Querying the data base for anomalous elements (variance and correlation) to identify anomalies and evaluating their spatial distribution
B. Querying for anomalous samples (sample correlation) and grouping them to identify anomalies and evaluating their spatial distribution
C. Assessing the spatial interdependency of samples deriving from a ore source

Commonly approach A is used. In this scenario the explorer intends to minimize the number of variables either by simply ignoring less meaningful elements according to the exploration target in mind. Another more sophisticated method is Factor Analysis. [Ref. 1, p. 11-13, Ref. 2, p. 163-168] The explorer using this method capitalizes on the correlation of elements to combine elements to new variables that explain the variance of the data set sufficiently. Both scenarios require an a priori knowledge about the nature of the target, either for justifying what elements should be considered or in the latter case the combinations of elements has to be meaningful to be interpreted for addressing the anomaly target. Further methods exist [Ref. 1, p. 4, Table 1] but all methods are variance driven and emphasis is given to element concentrations. Hence all methods of that category performing poor to detect weak element anomalies.

Approach B is represented by cluster analysis. [Ref. 2, p. 169-170] Samples are arranged in groups according to their similarity or dissimilarity without interference by the explorer. Dissimilarity is computed by a statistical distance measurement between multivariate samples. Processes known as Mahalanobis distance [Ref. 2, p. 163, 170, Ref. 3. p. 6] or Euclidean distance are applied. Similarity measures are usually Pearson correlation or other non-linear correlation among multivariate samples. All variables are considered simultaneously. Hence cluster analysis is unbiased, but not very robust statistically. The explorer has to predefine the number of groups and set thresholds for the group criteria that have significant impact on the assignment of samples to groups. The explorer faces the cumbersome task to determine the optimal number of groups to detect anomalies.

Approach C is relatively new and is still in an exploratory stage. A multivariate data matrix (sample) is considered in the spatial relationship to another multivariate matrix. The spatial distance between the samples is used to model the change in multivariate parameters that indicate the interdependency of samples and reflects their lineage. The method used is known as multivariate semivariogram and is ideal for recognizing the change of variables as a function of distance from a contamination or anomaly source without the presence of second order sources. The interpretation of the semivariogram becomes difficult if second order sources interfere which is often in a geological settings. The semivariogram requires a high sample density which is most often not met in the first steps of an exploration program.
References: [Ref 1] Household Surveys in Developing and Transition Countries: Design, Implementation and Analysis Chapter 18, Multivariate methods for index construction, Savitri Abeyasekera, Statistical Services Centre, The University of Reading, Reading, U.K.

[Ref 2] The Interpretation of Regional Geochemical Survey Data, Grunsky, E. C, In “Proceedings of Exploration 07: Fifth Decennial International Conference on Mineral Exploration” edited by B. Milkereit, 2007, p. 139-182

[Ref 3] Identifying Geochemical Anomalies, K. G. McQueen, Department of Earth and Marine Sciences Australian National University, ACT 0200, www.crcleme.org.au/Pubs/guides/gawler/a7_id.anomalies.pdf

### BRIEF SUMMARY OF THE INVENTION

The present invention is a method to identify sources of element anomalies and contaminations of collected and assayed rock-, soil-, sediment and organic matter samples. In one aspect of the invention this method is usable for quality control of mass produced material (e.g., concrete, plastics, technical products etc.) or to detect contamination of the environment.

In one aspect of the invention the method processes multivariate data by combining variance+correlation of variables (dissimilarity), sample correlation (similarity) and spatial sample interdependency in three steps, which encompasses three investigation levels (variable, sample, spatial sample distribution). Due to its holistic approach the anomaly identification is very robust.

The proposed method processes multivariate data without reducing the number of variables, hence preserves the information provided, but offers an unbiased simple two step anomaly identification that combines aspects of the conventional data query, sample comparison and sample interdependency. The method is not conforming to the overall goal of simplification of data by reducing the number of variables.

For instance the method addresses the detection of weak anomalies and it provides indications to the nature of the anomalies. The method is ideal for the appraisal of the resource potential of an undeveloped property due to its not required a priori knowledge.

In one aspect the invention features a method for identifying anomalies by determining the similarity and dissimilarity of samples in a sequenced fashion and assesses the spatial interdependency among samples.

In one embodiment of the invention multivariate data is organized in a multivariate data matrix or vector (geosignature) that is assigned to and is unique for every sample.

The similarity and dissimilarity computation of samples is based on the geo-signature input data.

Dissimilarity computation (for example Mahalanobis distance) produces first order anomalous samples and is followed up by similarity computation (linear or not linear multivariate correlation) that identifies second order anomalous samples.

In one embodiment of the invention the spatial interdependency of samples is investigated and shape, extent and spatial decline of sample similarity is used to interpret the nature of the first order anomaly.

The invention addresses the poor performance of conventional data interpretation for detecting weak anomalies and their biased approach (paragraph [0004]), the low robustness of the cluster analysis (paragraph [0005]). Although the presented method uses some computational elements of cluster analysis, it refrains from the grouping procedure and offers a simple and effective spatial interpretation about the nature of the anomaly.

### BRIEF DESCRIPTION OF THE SEVERAL VIEWS OF THE DRAWING

FIG. 1: The Visualized Geo-Signature of a Sample Composed of 47 Elements

Elements are plotted along the x-axis in a fixed order for all samples. The concentration of the elements is recorded in z-scores, or distances in standard deviation off the mean, along the y-axis. The chart is the geo-signature of the sample and depicts the element concentration and element distribution pattern of the sample. The element distribution, pattern is the “up and down” performance of the chart. For example the depicted chart can be understood as the visualization of the multivariate vector of the sample.

FIG. 2: Work Flow Chart

This work flow chart illustrates the steps required to use the presented method. The details of the steps are explained in the detailed description. The sequence of work goes from the top to the bottom of the page. “Source” stands for a source of element concentration anomalies and/or abnormal element distribution pattern. In this example a “source” is an ore body, but could be understood as any other anomalous entity. An exploration target is an inferred area encompassing all abnormal samples first and second order and the “source”.

FIG. 3: Similarity in Element Distribution Pattern

The dashed chart displays the geochemical signature of a sample that is diluted to a fourth of element concentrations compared to a sample depicted by the solid chart.

Both samples display the same element distribution pattern. The “ups and downs” are identical but the magnitude of the dashed chart is only one fourth the magnitude of the solid chart. In other words, both samples must have originated from the same source and the dashed-line sample is the diluted offspring. Both samples are identical in regards to their similarity. Similarity is the Pearson correlation, but any other correlation measure can be used.

FIG. 4: Super-Positioning of Mahalanobis Distance for Element Concentration and Mahalanobis Distance for Element Ratios

This example depicts the location of stream sediments collected from creeks. Black squares indicate known mineral occurrences. The Mahalanobis distance based on element concentration is plotted as circles. The sizes of the circles indicate the value of the Mahalanobis distance. Mahalanobis distance based on the ratio percentage between elements is displayed in stars in the appropriate size. Both Mahalanobis distances are staked for each sample.

FIG. 5: Similarity Among Prospective Anomalous Sample and Sediments

This plot indicates the Pearson correlation coefficient at the sample location. Different symbols are used that correspond to the grade of similarity. Pearson correlation of 1.0 is highest. Black squares indicate known mineral anomalies. Note the decreasing similarity of the geo-signature of samples with increasing distance to the prospective sample. This sample configuration is a classical fan shaped dilution and indicative of a “source” upstream.

FIG. 6: Similarity Among Ore Rock Sample and Rock- and Sediment Samples

The map shows sample locations of rock and sediments. Samples along the creek are stream sediments, all other samples are rock samples. The grade of similarity (by example Pearson correlation) among all samples and the ore rock sample is depicted in different symbols. The ore rock sample is noted by a self-correlation coefficient of 1.0.

FIG. 7: Idealized Sequence of Samples

The map displays the idealized sequence of samples downstream from a “source”, shown as a black square. Samples close to the “source” exhibit a high dissimilarity value (e.g. Mahalanobis distance) and are anomalous samples of first order. Those samples are shown as a star *. They occur spatially clustered and are therefore prospective samples. The extent of the spatial cluster delineates the primary prospecting area, displayed as the oval.

Anomalous samples of first order are followed downstream by anomalous samples of second order in FIG. 7 shown as circles O. Both sample categories are highly similar in their variable- (e.g. element-) distribution pattern of their geo-signature. Both samples originate from the same source and share the same lineage. Second order anomalous samples are more distant to the “source” and therefore their geo-signature is diluted but still similar to first order anomalous samples. Dilution in this context is understood as a geo-signature reduced in variable magnitude and the variable distribution pattern is altered to a lesser degree.

Eventually further downstream the similarity among samples and anomalous samples of first order are diminished. The geo-signature of those ordinary samples represents the surrounding lithology. The samples are depicted as squares.

FIG. 8: Wrapped Geo-Signature in a Spider Diagram

The diagram shows the geochemical signature as solid and dashed lines for two samples. The elements are plotted at the circumference of the diagram. The z-scores or distance in standard deviations from the mean for element concentrations are plotted on the spider web.

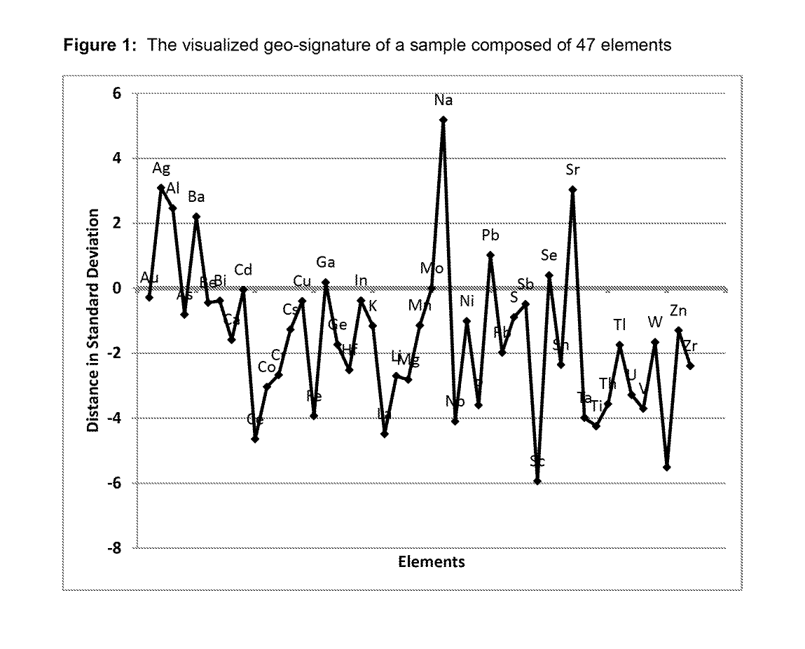

The diagram is a wrapped version of the x-y diagram used for portraying the geo-signature. (FIG. 1). For a similarity measure only the shape of the depicted area, and not its size underneath the chart is of interest.

To avoid the size factor, the plotted element concentrations can be replaced by element ratios.

### DETAILED DESCRIPTION OF THE INVENTION

The invention relates to a method that computes the dissimilarity and similarity for example of rock-, soil-, sediment and organic matter samples to identify sources of abnormal element concentration and element distribution (from now on called “source”). “Sources” may be ore bodies, contamination, product deficiencies or anomalies of other causes in the natural and technical environment. “Sources” are almost never abnormal in just one variable rather are multivariate anomalous.

For example in geochemical exploration the genesis of ore bodies is understood as a multi-element affair that culminates in the formation of a multivariate anomalous “source”. Multivariate anomalous “sources” are anomalous due to extreme variance of variables and/or abnormal variable correlation in respect to the general lithology. Dissimilarity computation detects abnormal samples that are perceived as proxy for “sources”. One embodiment of the invention for example addresses extreme element variance and abnormal element correlation simultaneously by computing the dissimilarity between samples. For instance the Mahalanobis distance as a multivariate outlier detection procedure may be used.

Ore bodies are anomalies, they are rare and they are dissimilar to the regional geology. The weathering of ore bodies to rock, soil and sediment and their subsequent mixture with the surrounding material generates to some degree a dilution and alteration of their chemical composition. In this process element concentrations are affected more than element distribution pattern (element correlation). Hence samples of similar element distribution pattern, regardless of their element concentration do originate from the same source. One embodiment of the invention among others addresses this situation by computing the similarity among samples. The similarity of samples is computed by the Pearson correlation, but any other correlation measure can be used. The similarity of geo-signatures rests on element distribution pattern and not on element concentrations.

The extent of ore deposits is locally constrained, but the extent of country rock composed of similar geology is usually not. The same applies to the weathered products of an ore body. One embodiment of the invention capitalizes on this condition. It considers the shape, extent and spatial degree of similarity among samples to deduce the nature of the anomaly. Samples that have a similar anomalous geo-signature and cluster are good indicators for “sources”. Anomalous samples that exhibit a fan shaped sample distribution of declining similarity away from the “source” are likewise good indicators.

Dissimilarity and similarity are fundamentally different computations. Dissimilarity looks for extremes, while similarity acknowledges relationship. The computation of dissimilarity and similarity between and among samples rests on the geosignature that is unique for every sample. The geosignature for example can be comprised of a multivariate matrix or vector with a fixed order of variables and their measurements. The geosignature may be visualized as a chart constructed upon the fixed order of elements and their abundances in an x-y-diagram (FIG. 1) or spider diagram (FIG. 8).

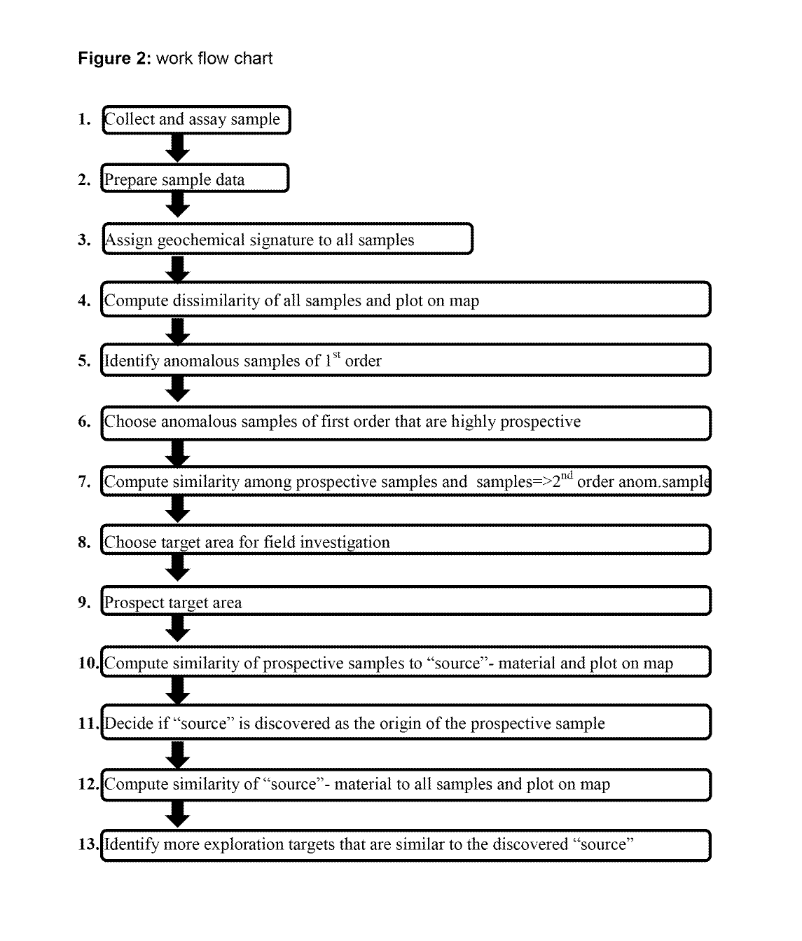

As an example FIG. 2 shows the steps of the invented method in the work flow chart for stream sediment analysis. Although the work flow chart indicates numerous computation of similarity and dissimilarity, both are only computed ones and saved as similarity matrix for samples and a vector or listing of dissimilarity for all samples. Computation in this context means picking the appropriate results from the matrix or listing. The method is comprised of the following steps:

Step 1: Samples are collected and assayed for the maximal number of elements available. The most accurate assay method applicable for all elements should be used.

Step 2: The element concentrations is standardize by computing z-scores for each sample to mitigate different element concentration magnitudes.

Step 3: The geo-signature to each sample is assigned. The geo-signature is comprised of all the variables and geo-referenced. An elements are organized for example in the form of a multivariate matrix or vector, where variables are arranged in a fixed order. For example the sequence of variable recordings can follow the alphabetic. The sequence must be consistent for all samples. Recordings can be concentrations, velocity, etc. The sample unique geo-signature may be visualized as a chart of all available elements recorded in a constant sequence along the x-axis of a diagram versus the corresponding element concentrations plotted in z-scores or distances in standard deviation from the mean along the y-axis as it's shown in FIG. 1. The geo-signature can also be displayed as an area shape in a spider diagram, seen in FIG. 8. The method does not require displaying the geo-signature of a sample graphically for computation purposes.

Step 4: The dissimilarity of the geosignatures for all samples is computed. For example the Mahalanobis distance or any other can be used. This measure detects outlier samples that have extreme element concentrations and/or abnormal element distribution pattern. Mahalanobis distance is calculated according to:

dS(x, y)=√(x−y)tS−1(x−y) and considers the covariance matrix S and vectors x and y.

Here written for a 2-dimensional space that can be expanded to an n-dimensional in the application. The n-dimension is determined by the number of elements used. All samples are plotted in a GIS environment displaying their Mahalanobis distance. One embodiment of the invention computes the Mahalanobis distance of samples based on their multivariate element concentrations. Another embodiment of the invention may consider element ratios as input variables. Both Mahalanobis distance for each sample may be displayed simultaneously by symbols in a stacked fashion at the sample location, seen in FIG. 4. Multiple dissimilarity measures of a sample can be compounded in indices and plotted at the sample location.

Step 5: The Mahalanobis distances of all samples may be arranged for example in a histogram. The histogram is used to determine the background. Samples with a Mahalanobis distance exceeding the background threshold are anomalous samples of first order.

Step 6: The spatial distribution of first order samples is assessed. For example spatial clusters of anomalous samples of first order indicate strong prospective targets close to the “source”. First order anomalous samples that show a directional gradient in Mahalanobis distance may display a dilution effect, increasing with distance to the “source”. Prospective anomalous first order samples are subject to further investigation in the following steps.

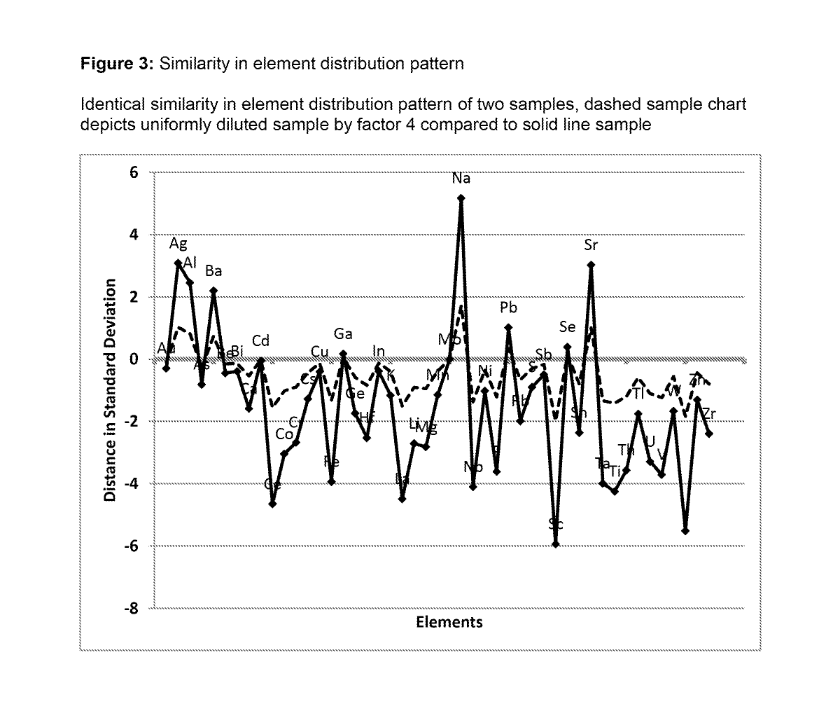

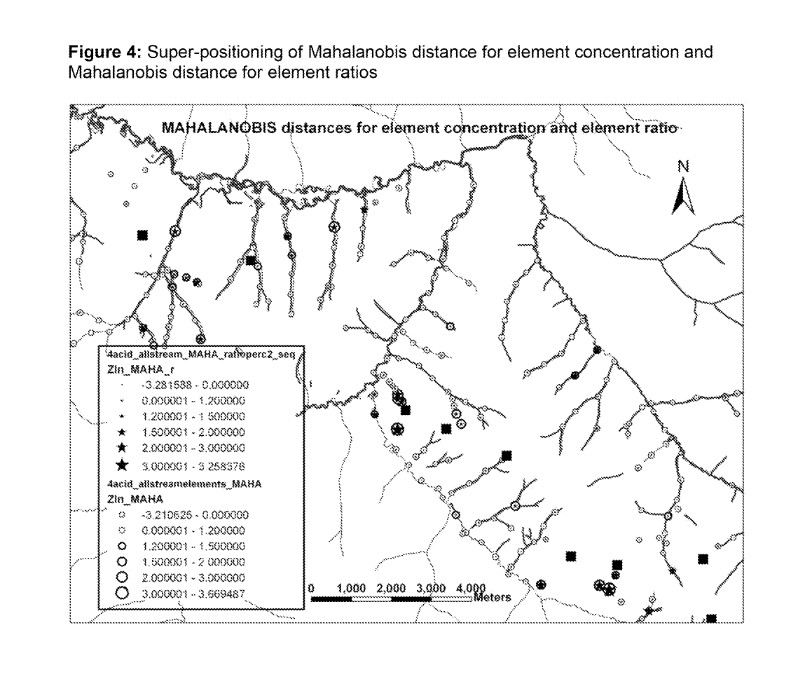

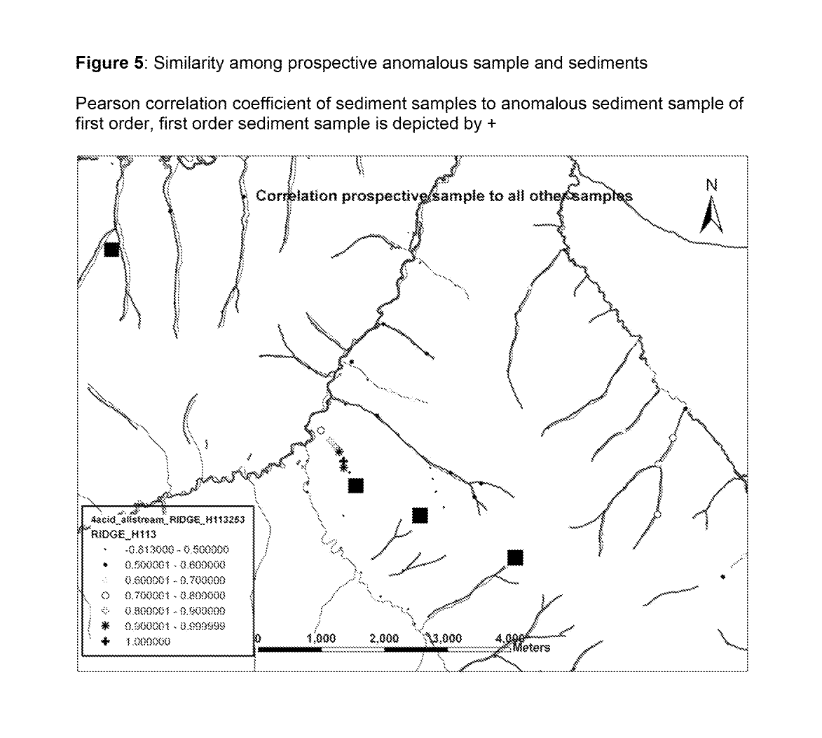

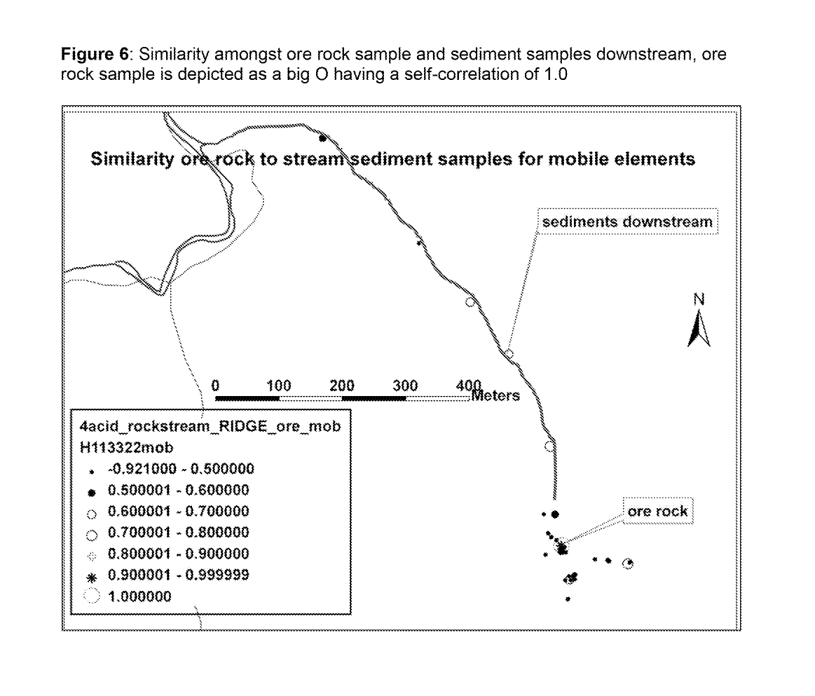

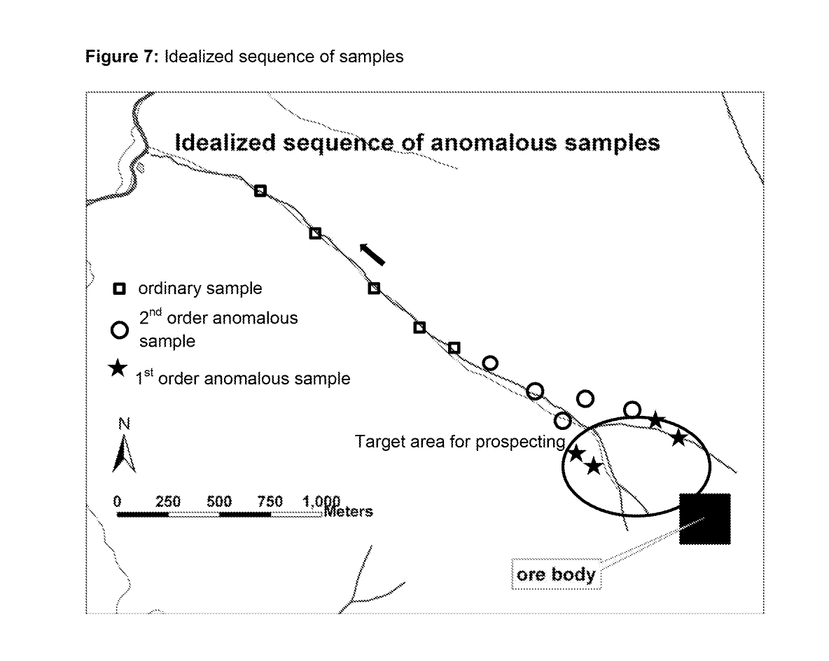

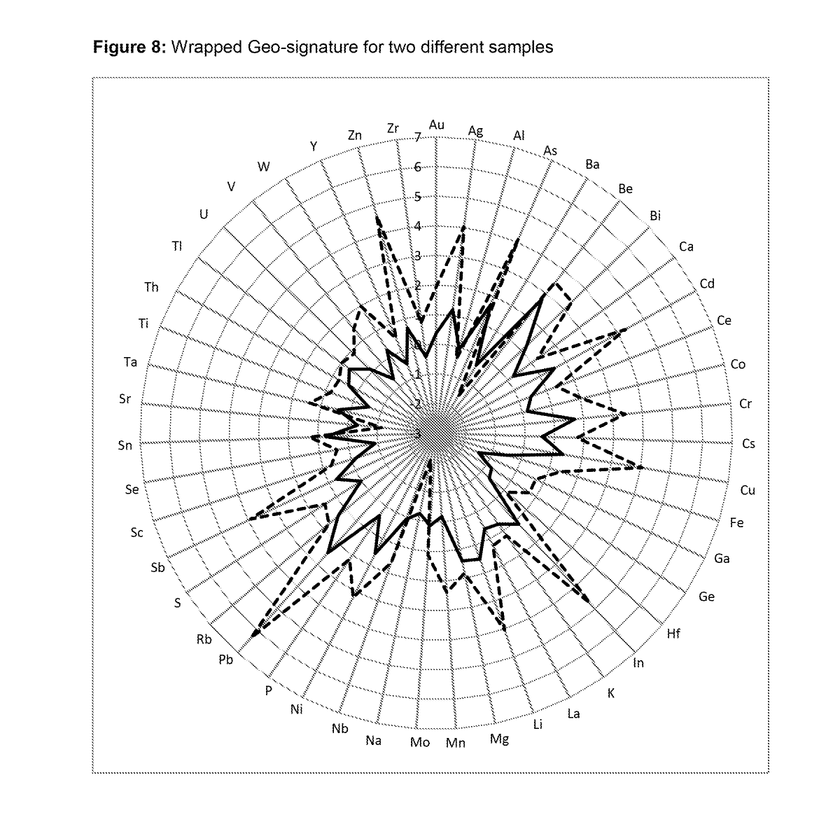

Step 7: In one embodiment of the invention among others, the similarity of all samples to the first order anomalous sample is computed and the similarity coefficient of the sample is plotted geo-referenced. This process may be repeated multiple times for each anomalous first order sample. For instance the Pearson correlation can be used, but any other similarity or correlation function can be applied. The Pearson correlation is a similarity measure that is very sensitive to changes of the element distribution pattern of the geochemical signature. The concentration of elements from one sample to another downstream changes quickly; however the element distribution pattern is altered at a much lower degree. FIG. 3 schematizes the effect dilution has on variable abundance and variable distribution pattern (variable correlation). The dashed-line chart, depicting the geo-signature of a sample diluted uniformly by a factor four. The “up and downs” of the element distribution pattern are identical, the sample are identical similar, although the element concentrations are not. The dashed-line sample originate from the solid line sample. They share the same lineage. In nature a uniform dilution of element concentrations is not observed. Therefore a Pearson correlation factor for example of 0.7 can be used as a threshold for the similarity of samples. Samples that exhibit for example a Pearson correlation coefficient above the threshold are deemed as similar to the anomalous samples first order and belong to the same lineage. Those samples are defined as anomalous samples of second order. In FIG. 5 for example the Pearson correlation coefficient of a sample in relation to the first order anomalous sample is depicted in symbols at the sample location. The same similarity procedure may also be applied to second order anomalous samples to produce third order anomalous samples.

The combination of dissimilarity- and similarity computation of the method reduces the impact that inaccurate variable readings have on the identification of anomalous samples of first and second order. Dissimilarity measures used for the invented method consider variable variance and covariance simultaneously (e.g. Mahalanobis distance). Variables that show unreliable readings or are dose to detection limits display artificially high variances and low covariance with other variables. They are not contributing substantially to the dissimilarity score of the sample. Therefore the samples are unlikely to be recognized as anomalous samples of first order by dissimilarity computation.
Likewise, erratic variables are rather destructive to consistent variable distribution pattern of the sample. The similarity among samples is kept low. Those samples are not qualifying as second order anomalous samples. The arrangement of dissimilarity- and similarity computation set out in the method limits the chance of creating anomaly targets based on unreliable variable readings.

Step 8: The spatial distribution of first and second order anomalous samples is assessed. A localized duster of first order anomalous samples merging into a fan of second order anomalous samples with gradually declining similarity away from the source may be a typical sample distribution for an ore body related “source”.
The example in FIG. 5 shows the decreasing similarity of samples downstream from the first order anomalous sample, which supports the perception of a locally confined source, auspicious for an ore body. In contrary a diffuse regional cluster of anomalous samples of first order and second order samples may be attributed to a regional change in geology.
The extent and shape of the similarity fan can be used to delineate the area of interest.

Step 9: The area of interest is prospected with the goal to discover the “source”. Rock-, soil-, and sediment samples are collected geo-referenced and assayed, using the same consisting assay procedure previously employed. The assay results are processed according to Step 1-3. Additional samples become part of the previous data base according to their entity. (E.g. sediments, rocks, soils)

Step 10: The similarity among all sediment- and rock samples and the first order anomalous sediment sample is computed by Pearson correlation for example. A follow up similarity computation may be performed among all sediment- and rock samples and a second order anomalous sample in the vicinity of the “source”. The “source” is identified if material collected from it exhibits high similarity to the first or second order anomalous sediment sample. The similarity coefficient for each rock- and sediment sample is plotted in symbols or numbers according to the corresponding sample location as seen in FIG. 6.
Obviously I can also compute the similarity among an identified “source” and all sediment samples that allows me to determine if the first order anomalous stream sediment sample derives from an ore source or from the benign country rock. The similarity between different phases of material like rock and sediments is naturally a magnitude lower than for the similarity of material belonging to the same entity. Lower similarity thresholds are to be expected.

In another embodiment of the invention the different mobility of elements is considered, which determines to what degree elements are able to migrate from one phase to another. Mobile elements have a tendency to be easier dissolved and transported into the sediments. The similarity computation can be based on groups of variables, deemed to be more appropriate for the situation.

Step 11: The source of the anomalous sediment of first order is found if said sediment is spatial related to the “source” and for example a high Pearson correlation between the first order and/or second order anomalous sediments and the “source” material is established. If no correlation is identified or no spatial relationship is conceivable the anomalous sediments are either a random anomaly or the “source” was not found yet.

Step 12: The similarity of identified “source” material (e.g. ore rock samples) and all samples (rock, soil and sediments) is computed. The similarity coefficient for each sample is plotted at the sample location. FIG. 7 shows an example of an idealized sample distribution for “source”-derived stream sediments.

Step 13: Samples that show a high similarity to the “source” but are spatial not related to the discovered “source” may have derived from a yet undetected “source” that is similar to the identified “source”. One embodiment of the invention among others provides a measure to discover anomalous samples that may have been missed by all previous steps. Similarity computation is used as a cross reference. Spatial cluster of second order anomalous samples not spatially related to first order anomalous samples may indicate weak “sources” that are not detected in the initial steps. Those diluted “sources” are identified by a similarity measure for example linear correlation of the geo-signature. Those weak anomalies are usually not detectable by conventional exploration methods. The underlying concept was already explained in Step: 7 and FIG. 3.

Any dissimilarity and similarity computation can be used that fulfills the objective of the invention to identify first and second order anomalous multivariate samples and considers their spatial interdependency. For instance the Mahalanobis distance can be based on element concentration or element ratios, or considers only mobile or immobile elements. Other dissimilarity measures that are capable of detecting multivariate outliers are available. Similarity measures used in the invention may be linear or nonlinear and may considering only subsets of the multivariate data.

If the method is used for quality control of mass produced material the spatial distribution pattern may be substituted by a time stamp referring to the time the material was produced. In this context neighboring products are such that were manufactured at around the same time. A cluster of anomalous and similar “faulty” material produced in sequence represents a systematic failure in the production and is not a random occurrence.

One embodiment of the invention among others displays the geo-signature in a spider diagram that creates an area underneath the chart. The multivariate geo-signature is wrapped around as seen in FIG. 8.
Each area shape is unique and it is just another representation of the geo-signature of a sample as seen in FIG. 1.
A software program can be used to recognize similar shapes regardless of the size of the area. Similar shapes (samples) cluster in histogram groups. Shapes that do not belong to a cluster or are only found in clusters of low membership are dissimilar and are outliers. Those qualify as anomalous samples of first order. If the identification of outliers cannot be solved graphically by software a statistical cluster analysis may do.

In another embodiment of the invention, cluster analysis can complement the Mahalanobis distance in Step: 4 of the work flow chart in FIG. 2.
Each sample is grouped according to its dissimilarity by a multivariate cluster analysis. The dissimilarity computation is based on element ratios.
Cluster analysis sorts samples according to similar element distribution pattern and categorizes them in sample clusters. Samples that belong to a cluster of low membership are anomalous and are recognized as anomalous samples of first order. All the following steps are analog the work flow chart in FIG. 2.

The invention claimed is:

1. A method for detecting anomalous objects or observations (referred to as samples) and assessing the nature thereof, assigning a geo-signature to every sample, determining the similarity and dissimilarity of samples' geo-signatures, evaluating the spatial distribution of similar samples, is comprised of the following operational phases:
1. a first phase for identifying the first order statistical anomalous samples,
2. a second phase for identifying the second order statistical anomalous samples,
3. a third phase considering the spatial distribution of similar first and second order anomalous samples for assessing their genesis,
4. a fourth phase for validating the interdependency, also spatially among the source of the anomaly and the first and second order samples,
5. a fifth phase for recognizing weak anomalies by identifying second order anomalous samples that are spatially separated but similar to the source of the anomaly said in the fourth phase.

2. A method that is incorporating all data variables and processes multivariate data simultaneously and unbiased, independent from the nature of the anticipated target.

3. A method according to claim 1, wherein said method is consisting essentially of two computation performances to generate first and second order anomaly targets and a final spatial evaluation stage: first; the computation of dissimilarity between entities and second; the computation of similarity among entities and finally assessing their spatial relationships.

4. A method according to claim 1, wherein said geo-signature is multivariate data organized in a multivariate data vector with constant structure and a fixed order of variables that is assigned to and is unique for every sample in its location.

5. A method according to claim 1, wherein said geo-signature is visualized by a chart in an x-y-diagram and/or an area shape in a spider diagram displaying the distribution pattern of the variables.

6. A method according to claim 1, wherein the dissimilarity and similarity of geo-signatures is computed by shape analysis of the area encompassed by the chart in a spider diagram

7. A method according to claim 1, wherein said geo-signature is used for determining first the dissimilarity and secondly the similarity of samples, processing all variables of the data matrix simultaneously.

8. A method according to claim 1, wherein said determining the similarity of samples' geo-signature is a correlation among geo-signatures, allowing for the recognition of similar samples based on variable distribution pattern, regardless of the magnitude of variables of the geo-signature.

9. A method according to claim 8, wherein said recognition of similar samples regardless of the magnitude of their geo-signature is aimed for identifying anomalous samples highly diluted and therefore not recognized by conventional methods.

10. A method according to claim 1, wherein said method is ideal but not restricted to geochemical exploration for ore sources using rock-, soil-, sediment- and organic material-samples.

11. A method according to claim 10, wherein said method intended for applying in geochemical exploration has a robust target recognition comprising of:
1. element correlations of the data set (Dissimilarity)
2. element concentrations of the data set (Dissimilarity)
3. element distribution pattern of the samples (Similarity)
4. spatial distribution of samples (shape, extent, interdependency)

12. A method according to claim 3, wherein the combination of said computation of similarity and dissimilarity among and between geo-signatures and said spatial evaluation is designed for down weighing the contribution of inaccurate and unreliable variable readings for target identification.
